(19) United States
(12) Patent Application Publication
(10) Pub. No.: US 2005/0250821 A1
(43) Pub. Date: Nov. 10, 2005

(54) Quaternary ammonium compounds in the treatment of water and as antimicrobial wash

(76) Inventors: Vincent Sewalt, West Des Moines, IA (US); Sally Ann Moore, Ankeny, IA (US); John A. Greaves, Ankeny, IA (US)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/107,281

(22) Filed: Apr. 15, 2005

Related U.S. Application Data
(60) Provisional application No. 60/562,822, filed on Apr. 16, 2004.

(51) Int. Cl. A61K 31/44; A61K 31/14; C02F 1/68

(52) U.S. Cl. 514/358; 514/642; 210/749

(57) ABSTRACT

Low levels of quaternary ammonium compounds are effective antimicrobial agents in drinking water and potentiate the antimicrobial power of organic acids used as antimicrobials for such purposes. The method is effective against both Gram (−) and Gram (+) bacteria, including but not limited to, Salmonella sp., E. coli, Campylobacter sp. as Staphylococcus sp. and Listeria sp. The combination of quaternary ammonium compounds and one or more organic acids can also be effectively used as antimicrobial washes for fruits, vegetables, meat, and animal carcasses.

### BACKGROUND OF THE INVENTION

1. Field of the Invention

The invention relates generally to the use of quaternary ammonium compounds as inhibitors of the growth of microorganisms and, more particularly to the use of quaternary ammonium compounds, such as cetylpyridinium chloride, alone and in combination with one or more organic acids to prevent the growth of pathogenic microorganisms in animal water supplies and as antimicrobial washes for fruit, vegetables, meat and animal carcasses.

2. Background of the Prior Art

Quaternary ammonium compounds are cationic surface active agents which have been shown to have antimicrobial effects against a number of bacteria present in the human mouth. Cetylpyridinium chloride, in particular, has been used in over-the-counter products such as lozenges, mouthwashes, and toothpastes for longer than 50 years. More recently, cetylpyridinium chloride has been used as a wash for reducing microbial contamination of fruits, vegetables, and meat.

An existing challenge in animal husbandry is the contamination of feed and drinking water with pathogenic and spoilage microorganisms. The contaminating organisms can adversely affect the health and growth of the animals and can also contaminate the animal products intended as human food products. Existing antimicrobial treatments for animal feed and drinking water include formaldehyde and organic acids, most commonly propionic acid.

According to the literature, cetylpyridinium chloride is able to prevent the attachment of or remove, if currently attached, Salmonella organisms from poultry tissue. (U.S. Pat. No. 5,366,983, Lattin, et al.; Kim, J. W., and M. F. Slavik. 1996. Cetylpyridinium chloride (CPC) treatment on poultry skin to reduce attached Salmonella. J. Food Protection 59:322-326). It has also been found effective as an antimicrobial wash for fruits and vegetables. (Lukasik, J., M. L. Bradley, T. M. Scott, M. Dea, A. Koo, W. Y. Hsu, J. A. Bartz, and S. R. Farrah. 2003. Reduction of Poliovirus 1, bacteriophages, Salmonella Montevideo, and Escherichia coli O157:H7 on strawberries by physical and disinfectant washes; Tran, T. T., R. N. Matthews, C. R. Warner, and S. J. Chirtel. 2002. Effectiveness of cetylpyridinium chloride and commercial vegetable wash preparations on the viability of indigenous bacterial flora of selected fresh produce. Poster Abstract L-10 presented at “FDA: Building a Multidisciplinary Foundation”. 2002 FDA Science Forum, Feb. 20-21, 2002, Washington, D.C.) However, this requires relatively high concentrations, as high as 0.1% or 1000 ppm (e.g., Kim and Slavik). It has also been shown that produce washes containing cetyl pyridinium chloride (CPC) can reduce the number of Salmonella, and E. coli, organisms on fresh fruit and vegetables (Lukasik et al., 2003; Tran et al., 2002), in both cases again requiring a CPC concentration of 0.1% (1000 ppm). Cationic surfactants will also interact with the lipopolysaccharide layer of the bacterial cell membrane leading to the disruption of the membrane and eventual cell death. Organic acids, on the other hand, are capable of entering bacterial cells in an undissociated form, i.e., at relatively low pH. Once inside the more neutral environment within the bacterial cells, the organic acid dissociates resulting in the release of protons that destabilize internal membranes and that can only be removed from the cell by the proton pump, a process that depletes the cell's energy.

A need exists for effective antimicrobial treatments for animal drinking water that may be used either independently or in combination with existing antimicrobial treatment methodologies to improve performance or effectiveness, reduce cost, or combinations of the same.

### SUMMARY OF THE INVENTION

The invention consists of the use of quaternary ammonium compounds, alone and in combination with one or more organic acids to prevent the growth of pathogenic and spoilage microorganisms in animal drinking water. Quaternary ammonium compounds, particularly cetylpyridinium chloride, are shown to have efficacy as an antimicrobial treatment for animal drinking water both by itself and in combination with existing treatments such as or organic acids. The concentration of CPC when combined with organic acids shown to be effective in inhibiting the growth of Gram(+) and Gram(−) bacteria is as low as about 0.1 part per million (ppm), up to at least 1000 ppm, and preferably between about 2.0 and about 50 ppm. CPC potentiates the antimicrobial activity of organic acids up to five-fold and is combined with organic acids to provide an effective antimicrobial treatment at a reduced cost. CPC is effective against the pathogenic microorganisms Salmonella, Campylobacter, Listeria, and Staphylococcus, as well as E. coli. CPC is also effective against yeast, e.g., Candida castellii. The invention would also be useful as an antimicrobial wash for fruits, vegetables, meat and animal carcasses to reduce the load of pathogenic microorganisms on such products and, in particular, to reduce the potential for cross-contamination of carcasses during processing.

An object of the present invention is the use of quaternary ammonium compounds as antimicrobial agents in the treatment of water, including but not limited to human or animal drinking water.

Another object of the invention is the use of quaternary ammonium compounds in synergistic combination with organic acids as antimicrobial agents in the treatment of water, including but not limited to human and animal drinking water.

A further object of the present invention is the use of quaternary ammonium compounds in synergistic combination with organic acids as antimicrobial washes for fruits, vegetables, meat and animal carcasses.

These and other objects of the invention will be made known to those skilled in the art upon a review and understanding of this specification, the associated figures, and the appended claims.

### BRIEF DESCRIPTION OF THE FIGURES

FIG. 1 is a chart of the inhibition of Salmonella Enteritidis by CPC at three treatment levels in a microtiter plate assay using absolute values for optical density measurements.

FIG. 2 is a chart of the inhibition of Staphylococcus aureus by CPC at three treatment levels in a microtiter plate assay using absolute values for optical density measurements.

FIG. 3 is a chart of the inhibition of Candida castellii by CPC at three treatment levels in a microtiter plate assay using absolute values for optical density measurements.

FIG. 4 is a chart of CPC potentiation of propionic acid in inhibiting Salmonella Enteritidis in a 3×3 experimental design.

FIG. 5 is a chart of CPC potentiation of propionic acid in inhibiting E. coli O157:H7 in an abbreviated 4×4 experimental design.

FIG. 6 is a chart of CPC potentiation of propionic acid in inhibiting Staphylococcus aureus in a 4×4 experimental design.

FIG. 7 is a chart of CPC potentiation of propionic acid in inhibiting Listeria monocytogenes in an abbreviated experimental design.

FIG. 8 is a chart of CPC potentiation of mixed organic acids in inhibiting Salmonella Enteriditis.

FIG. 9 is a chart of CPC potentiation of mixed organic acids in inhibiting E. coli O157:H7.

FIG. 10 is a chart of CPC potentiation of acetic acid in inhibiting Salmonella Enteriditis.

FIG. 11 is a chart of CPC potentiation of acetic acid in inhibiting Staphylococcus aureus.

FIG. 12 is a chart of the effect of intermediate and high dosages of a commercially available liquid water acidifier designated KS and KS with 3×CPC (KS w/CPC) over time on coliforms in water collected from a commercial swine farm.

FIG. 13 is a chart of the dose response to 6-h exposure to KS water acidifier as influenced by inclusion rate of cetylpyridinium chloride in reducing coliform counts in drinking water obtained from a commercial swine farm.

FIG. 14 is a chart of the effect of the intermediate dose of KS water acidifier alone and KS with 2× and 3×CPC over time in reducing coliform counts in drinking water obtained from a commercial swine farm.

### DETAILED DESCRIPTION OF PREFERRED EMBODIMENTS

Quaternary ammonium compounds are cationic surface active agents and those compounds included in the present invention are selected from the list that includes alkylpyridinium, tetra-alkylammonium, and alkylalicyclic ammonium salts.

Coliforms are aerobic or facultatively anaerobic, Gram-negative, non-sporeforming rods that ferment lactose.

Organic acids are organic compounds that are acids. Organic acids include acetic, benzoic, citric, formic, fumaric, lactic, propionic, and sorbic acids.

The following descriptions are supportive of the preferred embodiments of this invention, including the use of synergistic mixtures of organic acids and quaternary ammonium compounds to eliminate or retard growth of pathogenic bacteria, spoilage bacteria, and yeasts in water, including the cleaning of water lines, the use of treated water as drinking water, and the use of treated water as antimicrobial wash. The combination of one or more organic acids with quaternary ammonia compounds such as CPC results in lower dosages of either type of compound alone required to effectively stop and prevent microbial growth in each of these water-based applications.

Experiment 1

Five test organisms—Salmonella Enteritidis (ATCC 13076), Staphylococcus aureus (ATCC 25923), Candida castellii (field strain), and two laboratory mold cultures Aspergillus and Fusarium—were selected for use in the initial screening assays. Cetylpyridinium chloride (CPC) was obtained from Aceto Corporation. Bacterial cells were grown aerobically in Tryptic Soy Broth (TSB) for 24 h at 37° C. Yeast cells were grown in Potato Dextrose Broth (PDB) aerobically for 24 h at 37° C. Mold organisms were grown on Potato Dextrose Agar (PDA) at room temperature until sufficient sporulation was apparent. Test inocula were prepared to achieve a 10⁶ cfu/ml suspension of bacterial cells and a 10⁵ cfu/ml suspension of both yeast and mold spores. A Petroff Hausser counting chamber was used to determine the level of inoculum.

Poison Agar Assay. To evaluate the efficacy of CPC in mold inhibition, a poison agar assay was utilized. Sterile PDA was treated with CPC to achieve final treatment levels of 500, 1000 and 1500 ppm (w/v) in sterile agar. Sterile paper disks were impregnated with 10⁵ mold spores/ml, allowed to dry under a laminar flow hood and disks, in triplicate, were aseptically placed onto the treated agar surface. Control plates consisted of untreated agar with paper disks impregnated with mold spores. All agar plates were incubated at 25° C. for 72 h. Percent inhibition was determined by measuring, in millimeters, the diameter of mold growth radiating from the paper disk.

Microtiter Plate Assay. To evaluate the efficacy of CPC in bacteria and yeast inhibition, a microtiter plate assay was utilized. An Optimax microtiter plate reader (Molecular Devices, Sunnyvale, Calif.) at 405 nm wavelength was used to measure the optical density of the suspension in each well. Plates were read kinetically over 24 h. The temperature was maintained at 35° C. All results reflect the average optical density measurements of four microtiter wells. CPC treatments were prepared (w/v) in sterile RO water resulting in active treatment levels of 2.5, 10.0, and 25.0 ppm. A 100 μl aliquot of test organism and a 100 μl aliquot of experimental treatment were dispensed into individual microtiter plate wells. Bacteria cultures were diluted in Nutrient Broth (NB) prior to inoculation into the wells due to cloudiness caused by the interaction of TSB medium and CPC. Positive controls (PC) consisting of 100 μl of test organism in NB or PDB and 100 μl of sterile water and a negative control (NC) consisting of 100 μl of NB or PDB (no organisms) and 100 μl of sterile water were also used.

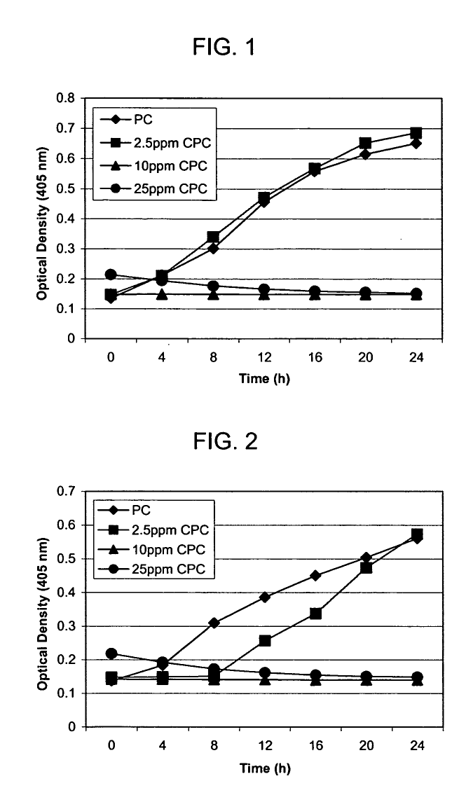

CPC was effective in inhibiting the growth of S. Enteriditis. FIG. 1 shows the Minimum Inhibitory Concentration (MIC) of CPC for inhibition of this bacterium to be 10 ppm. For comparative purposes, the MIC in this assay of 37% formaldehyde, a potent antimicrobial approved for Salmonella control in feed, is approximately 50 ppm.

Similarly, CPC completely inhibited the growth of S. aureus at a concentration of 10 ppm. FIG. 2 depicts the effects of all treatment levels on the growth of S. aureus. Quarternary ammonium compounds are very effective against Gram-positive bacteria and CPC inhibited the growth of this organism at the 2.5 ppm treatment level through the initial 8 hours of growth.

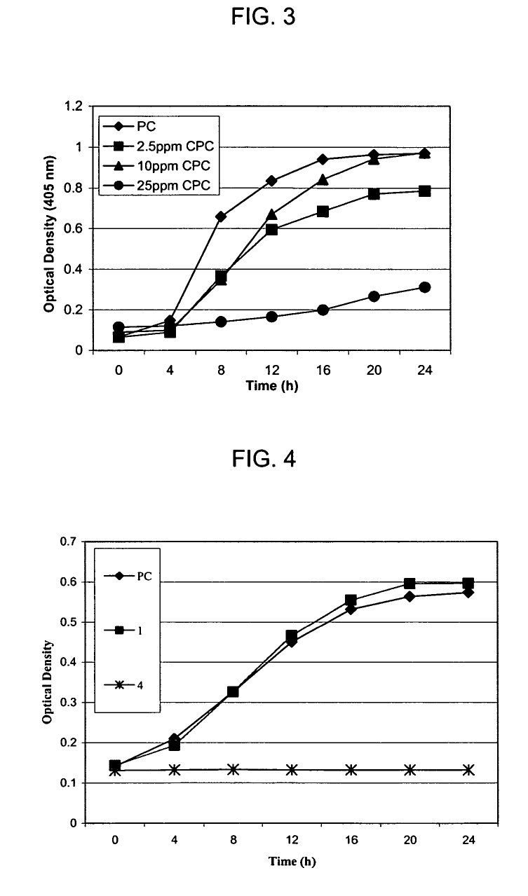

CPC was also effective in the inhibition of a yeast organism (FIG. 3), although an MIC of >25 ppm would be required for complete inhibition of C. castellii growth. Previous experiments resulted in complete growth inhibition of this yeast organism at 100 ppm.

In mold inhibition, CPC was more effective against Aspergillus than Fusarium. The MIC level for inhibition of an Aspergillus mold was <1000 ppm whereas the MIC for Fusarium mold was >1500 ppm. Table 1 compares the inhibition of Aspergillus and Fusarium from each treatment level as compared to an untreated control.

TABLE 1
Inhibition of Aspergillus and Fusarium (% of
control) due to treatment of CPC

| Mold/ | Treatment | | |
|---|---|---|---|
| Treatment | Level (ppm) | | |
| Level | 500 | 1000 | 1500 |
| Control | 0.0 | | |
| Aspergillus | 57.2 | 100.0 | 100.0 |
| Fusarium | 61.0 | 64.3 | 69.0 |

Efficacy of treatment with cetylpyridinium chloride (CPC) varied with type of microorganism. Overall, growth inhibition was higher for bacteria and yeast than for ftmgal organisms. Experiments determined the MIC of this compound for the specific microorganisms selected.

Experiment 2

Cetylpyridinium chloride (CPC) has been shown to be an effective inhibitor of microbial growth. Experiments were conducted to determine the synergistic effects of CPC in combination with propionic acid as an antimicrobial/pathogen reduction intervention in water.

Either a 3×3 or 4×4 matrix design was created to examine the effects of CPC and propionic acid against the growth of Salmonella Enteriditis, Staphylococcus aureus, E. coli O157:H7, and Listeria monocytogenes. The 4×4 matrix was either used in its entirety or in an abbreviated format as deemed appropriate by previous research conducted with similar organisms using a mixed organic acid blend.

Eight formulations were prepared using the incomplete 3×3 matrix design in Table 2. Eight combinations were prepared using 1, 5 and 10 ppm CPC and 50, 100 and 250 ppm propionic acid and given numeric identifications of 1-8. Positive and negative controls were included as described in Experiment 1.

TABLE 2
A 3 × 3 matrix design of 8 combinations of CPC and
propionic acid (formulas 1-8) to evaluated efficacy
against Salmonella.

| | Propionic Acid (ppm) | | |
|---|---|---|---|
| CPC (ppm) | 50 | 100 | 250 |
| 1 | 1 | 2 | 3 |
| 5 | 4 | 5 | 6 |
| 10 | 7 | 8 | |

Fifteen formulations were prepared using the incomplete 4×4 matrix design in Table 3. Fifteen combinations were prepared using 0, 1, 5 and 10 ppm CPC and 50, 100, 150 and 250 ppm propionic acid and given numeric identifications of 1-15.

TABLE 3
A 4 × 4 matrix design of 15 combinations of CPC and
propionic acid (formulas 1-15) to evaluate efficacy
against Staphylococcus, E. coli, and Listeria.

Propionic Acid (ppm)

| CPC (ppm) | 50 | 100 | 150 | 250 |
|---|---|---|---|---|
| 0 | 1 | 2 | 3 | 4 |
| 1 | 5 | 6 | 7 | 8 |
| 5 | 9 | 10 | 11 | 12 |
| 10 | 13 | 14 | 15 | |

Four test organisms—Salmonella Enteritidis (ATCC 13076), Staphylococcus aureus (ATCC 25923) E. coli O157:H7 (ATCC 35150) and Listeria monocytogenes (ATCC 15313)—were selected for use in these screening assays. Cetylpyridinium chloride was obtained from Aceto Corporation. Propionic acid was obtained from Kemin Americas, Inc (Des Moines, Iowa). S. Enteriditis, S. aureus and E. coli were grown aerobically in either Tryptic Soy Broth (TSB) or Nutrient Broth (NB) for 24 h at 37° C. L. monocytogenes was grown aerobically in Brain Heart Infusion Broth (BHI) for 24 h at 37° C. Test inocula were prepared to achieve a 10⁶ cfu/ml suspension of bacterial cells. A Petroff Hausser counting chamber was used to determine the level of inoculum.

To evaluate the efficacy of CPC and propionic acid combinations in bacterial inhibition, a microtiter plate assay was utilized. An Optimax microtiter plate reader (Molecular Devices, Sunnyvale, Calif.) at 405 nm wavelength was used to measure the optical density of the suspension in each well. Plates were read kinetically over 24 h. The temperature was maintained at 35° C. All results reflect the average optical density measurements of four microtiter wells. Treatments were prepared in sterile RO at the inclusion levels noted in Tables 1-2. A 100 μl aliquot of test organism and a 100 μl aliquot of experimental treatment were dispensed into individual microtiter plate wells. Bacteria cultures were diluted in Nutrient Broth (NB) prior to inoculation into the wells due to cloudiness caused by the interaction of TSB medium and CPC. BHI did not cause this effect and continued to be used for growth of L. monocytogenes. Positive controls consisting of 100 μl of test organism in NB or BHI and 100 μl of sterile water and a negative control consisting of 100 μl of NB (no organisms) and 100 μl of sterile water were also used.

Cetylpyridinium chloride was found to potentiate the antimicrobial effect of propionic acid. Figures and Tables 4 and 5 represent the effects of CPC and propionic acid in the inhibition of Gram-negative organisms. In Figure/Table 4, Formula 4 (5 ppm CPC/50 ppm propionic acid) was shown to completely inhibit the growth of Salmonella Enteriditis. Previous experimentation showed that 250 ppm propionic acid was sufficient to inhibit Salmonella growth and complete inhibition from Formulas 3 and 6 was expected. Due to the limitations presented by this matrix, a 4×4 design was used for further evaluations.

TABLE 4
CPC potentiation of propionic acid in inhibiting Salmonella Enteritidis
in a 3 × 3 experimental design

| | Hours | | | | | | |
|---|---|---|---|---|---|---|---|
| | 0 | 4 | 8 | 12 | 16 | 20 | 24 |
| Treatment | Optical Density | | | | | | |
| PC | 0.141 | 0.210 | 0.326 | 0.450 | 0.532 | 0.564 | 0.574 |
| 1 | 0.143 | 0.193 | 0.326 | 0.466 | 0.555 | 0.596 | 0.597 |
| 2 | 0.143 | 0.147 | 0.190 | 0.283 | 0.382 | 0.473 | 0.517 |
| 3 | 0.135 | 0.136 | 0.136 | 0.136 | 0.136 | 0.135 | 0.135 |
| 4 | 0.131 | 0.132 | 0.133 | 0.132 | 0.132 | 0.132 | 0.132 |
| 5 | 0.149 | 0.146 | 0.146 | 0.146 | 0.145 | 0.144 | 0.144 |
| 6 | 0.150 | 0.150 | 0.147 | 0.145 | 0.144 | 0.143 | 0.142 |
| 7 | 0.141 | 0.143 | 0.143 | 0.143 | 0.142 | 0.142 | 0.141 |
| 8 | 0.138 | 0.139 | 0.139 | 0.139 | 0.139 | 0.139 | 0.139 |

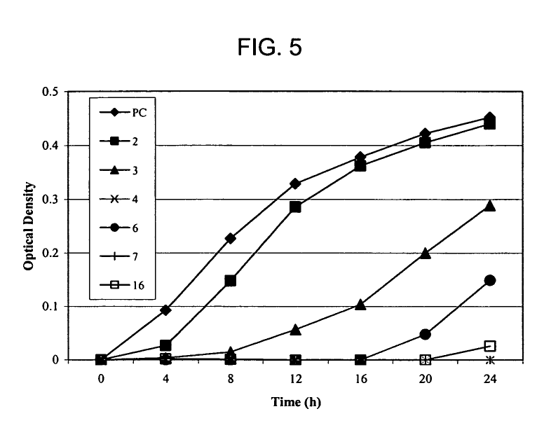

An abbreviated 4×4 design was used for the evaluation of E. coli O157:H7. In FIG. 5, Formula 7 (1 ppm CPC/150 ppm propionic acid) was shown to completely inhibit the growth of E. coli, whereas 150 ppm of propionic acid alone (Formula 3) inhibited growth for only 4 h. Formula 6 (1 ppm CPC/100 ppm propionic acid) inhibited growth for 16 h with no inhibition noted for 100 ppm propionic acid alone (Formula 2). Essentially, 1 ppm CPC was able to substitute 50 ppm propionic acid. As expected 250 ppm propionic acid treatment alone completely inhibited the growth of E. coli.

TABLE 5
CPC potentiation of propionic acid in inhibiting E. coli O157:H7
in an abbreviated 4 × 4 experimental design

| | Hours | | | | | | |
|---|---|---|---|---|---|---|---|
| | 0 | 4 | 8 | 12 | 16 | 20 | 24 |
| Treatment | Optical Density | | | | | | |
| PC | 0.000 | 0.093 | 0.227 | 0.329 | 0.378 | 0.422 | 0.452 |
| 2 | 0.000 | 0.027 | 0.148 | 0.286 | 0.362 | 0.405 | 0.440 |
| 3 | 0.000 | 0.004 | 0.015 | 0.057 | 0.104 | 0.200 | 0.289 |
| 4 | 0.000 | 0.001 | 0.000 | 0.000 | 0.000 | 0.000 | 0.000 |
| 5 | 0.000 | 0.042 | 0.202 | 0.331 | 0.370 | 0.385 | 0.418 |
| 6 | 0.000 | 0.000 | 0.000 | 0.000 | 0.000 | 0.048 | 0.149 |
| 7 | 0.000 | 0.000 | 0.000 | 0.000 | 0.000 | 0.000 | 0.000 |
| 9 | 0.000 | 0.000 | 0.000 | 0.000 | 0.000 | 0.000 | 0.000 |
| 10 | 0.000 | 0.000 | 0.000 | 0.000 | 0.000 | 0.000 | 0.000 |
| 11 | 0.000 | 0.000 | 0.000 | 0.000 | 0.000 | 0.000 | 0.000 |

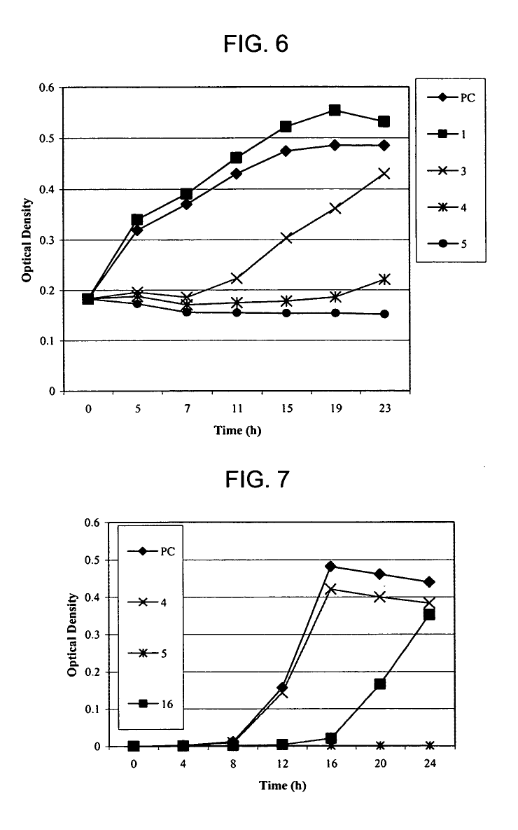

The ability of CPC to potentiate propionic acid in inhibiting two Gram-positive organisms was also evaluated. Previous experimentation had shown 250 ppm of propionic acid to inhibit the growth of S. aureus. This organism was screened against the full 4×4 matrix. The data presented in Table and FIG. 6 show that formula 5 (1 ppm CPC/50 ppm propionic acid) was able to completely inhibit the growth of S. aureus whereas 50 ppm propionic acid (Formula 1) resulted in no inhibition. A level of 150 ppm propionic acid (Formula 3) did not inhibit growth and, in this experiment, minimal growth was observed at an exposure of 250 ppm propionic acid (Formula 4). It could be postulated that 1 ppm CPC could substitute for approximately 200 ppm propionic acid.

TABLE 6
CPC potentiation of propionic acid in inhibiting Staphylococcus
aureus in a 4 × 4 experimental design

| | Hours | | | | | | |
|---|---|---|---|---|---|---|---|
| | 0 | 5 | 7 | 11 | 15 | 19 | 23 |
| Treatment | Optical Density | | | | | | |
| PC | 0.183 | 0.319 | 0.37 | 0.43 | 0.474 | 0.485 | 0.485 |
| 1 | 0.183 | 0.34 | 0.391 | 0.461 | 0.522 | 0.554 | 0.532 |
| 2 | 0.183 | 0.233 | 0.256 | 0.348 | 0.412 | 0.487 | 0.538 |
| 3 | 0.183 | 0.196 | 0.186 | 0.223 | 0.303 | 0.361 | 0.43 |
| 4 | 0.183 | 0.188 | 0.171 | 0.175 | 0.178 | 0.186 | 0.221 |
| 5 | 0.183 | 0.173 | 0.156 | 0.155 | 0.154 | 0.154 | 0.152 |
| 6 | 0.183 | 0.164 | 0.157 | 0.156 | 0.155 | 0.153 | 0.153 |
| 7 | 0.183 | 0.163 | 0.161 | 0.160 | 0.159 | 0.158 | 0.158 |
| 8 | 0.183 | 0.162 | 0.161 | 0.160 | 0.158 | 0.157 | 0.157 |
| 9 | 0.183 | 0.195 | 0.177 | 0.176 | 0.175 | 0.174 | 0.174 |
| 10 | 0.183 | 0.185 | 0.165 | 0.163 | 0.162 | 0.161 | 0.160 |
| 11 | 0.183 | 0.196 | 0.175 | 0.174 | 0.172 | 0.171 | 0.170 |
| 12 | 0.183 | 0.194 | 0.181 | 0.178 | 0.177 | 0.176 | 0.175 |
| 13 | 0.183 | 0.186 | 0.176 | 0.174 | 0.172 | 0.170 | 0.169 |
| 14 | 0.183 | 0.187 | 0.182 | 0.176 | 0.171 | 0.168 | 0.166 |
| 15 | 0.183 | 0.193 | 0.186 | 0.180 | 0.175 | 0.173 | 0.171 |
| NC | 0.183 | 0.183 | 0.179 | 0.178 | 0.177 | 0.176 | 0.176 |

The abbreviated 4×4 matrix design was used to determine CPC potentiation of propionic acid in the inhibition of L. monocytogenes. The data is presented in Table 7 and illustrated in FIG. 7. Formula 5 (1 ppm CPC/50 ppm propionic acid) was able to completely inhibit the growth of L. monocytogenes whereas levels of propionic acid at 250 ppm (Formula 4) did not affect the growth of this organism. In this case, it would appear that 1 ppm CPC could substitute for >200 ppm propionic acid.

TABLE 7
CPC potentiation of propionic acid in inhibiting L. monocytogenes

| | Hours | | | | | | |
|---|---|---|---|---|---|---|---|
| | 0 | 4 | 8 | 12 | 16 | 20 | 24 |
| Treatment | Optical Density | | | | | | |
| PC | 0.000 | 0.002 | 0.012 | 0.157 | 0.482 | 0.461 | 0.440 |
| 2 | 0.000 | 0.001 | 0.011 | 0.167 | 0.471 | 0.448 | 0.432 |
| 3 | 0.000 | 0.000 | 0.011 | 0.173 | 0.479 | 0.451 | 0.428 |
| 4 | 0.000 | 0.001 | 0.010 | 0.143 | 0.421 | 0.400 | 0.384 |
| 5 | 0.000 | 0.000 | 0.001 | 0.002 | 0.001 | 0.001 | 0.001 |
| 6 | 0.000 | 0.000 | 0.002 | 0.001 | 0.001 | 0.001 | 0.002 |
| 7 | 0.000 | 0.000 | 0.001 | 0.001 | 0.002 | 0.002 | 0.002 |
| 9 | 0.000 | 0.000 | 0.001 | 0.001 | 0.001 | 0.001 | 0.002 |
| 10 | 0.000 | 0.000 | 0.001 | 0.001 | 0.001 | 0.001 | 0.002 |
| 11 | 0.000 | 0.000 | 0.001 | 0.001 | 0.001 | 0.001 | 0.001 |

Low levels of CPC potentiated the antimicrobial power of propionic acid. The modes of action of each molecule appear to be complementary. It is likely that the interaction of CPC with the material cell membrane facilitates the entry of organic acids into the cell, resulting in a lower lethal dosage of the respective organic acid. A greater CPC potentiation of propionic acid was observed with Listeria and Staphylococcus, as quaternary ammonium compounds are very effective antimicrobial agents against Gram-positive bacteria. To effectively inhibit Gram(−) and Gram(+) bacteria, 1 ppm CPC can replace 50 or 200 ppm of propionic acid, respectively. These experiments show that CPC in combination with propionic acid will augment the reduction of pathogens and enhance food safety initiatives.

Experiment 3

Fifteen formulations were prepared using the incomplete 4×4 matrix design in Table 8. Fifteen combinations were prepared using 0, 1, 5 and 10 ppm CPC and 50, 100, 150 and 250 ppm of a mixture of organic acids product (Feed CURB®, Kemin Americas, Inc.) containing propionic acid, acetic acid, benzoic acid and sorbic acid, and given numeric identifications of 1-15.

TABLE 8
A 4 × 4 matrix design of 15 combinations of CPC
and mixed organic acids

Organic Acids (ppm)

| CPC (ppm) | 50 | 100 | 150 | 250 |
|---|---|---|---|---|
| 0 | 1 | 2 | 3 | 4 |
| 1 | 5 | 6 | 7 | 8 |
| 5 | 9 | 10 | 11 | 12 |
| 10 | 13 | 14 | 15 | |

Two test organisms—Salmonella Enteritidis (ATCC 13076) and E. coli O157:H7 (ATCC 35150)—were screened. Salmonella Enteritidis was grown aerobically in Nutrient Broth (NB) for 24 h at 37° C. E. coli was grown in NB for 6 h at 37° C. Test inocula were prepared to achieve a 10⁶ cfu/ml suspension of bacterial cells. A Petroff Hausser counting chamber was used to determine the level of inoculum.

Experiments were conducted to determine the synergistic effects of CPC in combination with mixed organic acids as an antimicrobial/pathogen reduction intervention in poultry and livestock water. To evaluate the efficacy of combinations of CPC and mixed organic acids in bacterial inhibition, a microtiter plate assay was utilized. An Optimax microtiter plate reader (Molecular Devices, Sunnyvale, Calif.) at 405 nm wavelength was used to measure the optical density of the suspension in each well. Plates were read kinetically over 24 h. The temperature was maintained at 35° C. All results reflect the average optical density measurements of four microtiter wells. Treatments were prepared in sterile RO at the inclusion levels noted in Table 1. A 100 μl aliquot of test organism and a 100 μl aliquot of experimental treatment were dispensed into individual microtiter plate wells. Bacteria cultures were diluted in Nutrient Broth (NB) prior to inoculation into the wells. Positive controls consisting of 100 μl of test organism in NB and 100 μl of sterile water and a negative control consisting of 100 μl of NB (no organisms) and 100 μl of sterile water were also used.

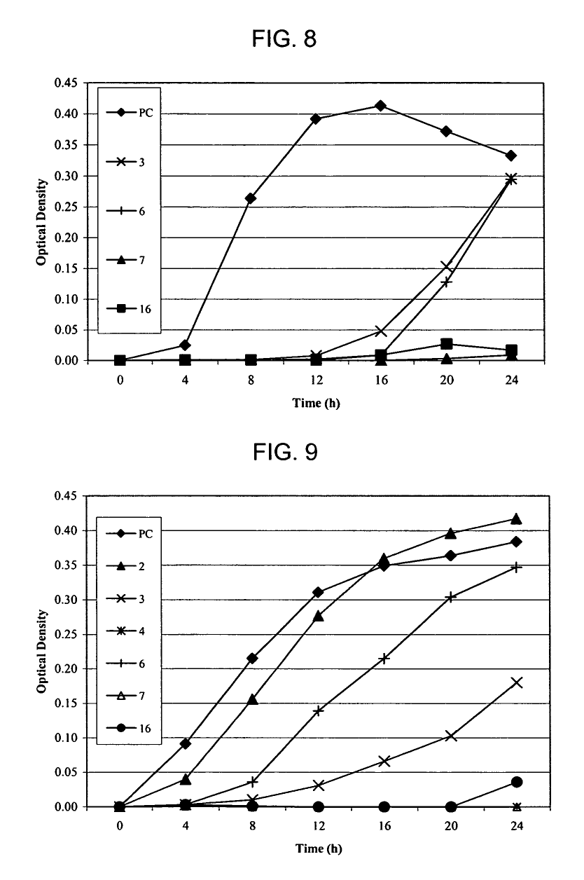

Cetylpyridinium chloride was found to potentiate the antimicrobial effect of mixed organic acids toward Salmonella and E. coli The results are shown in Table 9 and 10 and FIGS. 8 and 9. In FIG. 8, Formula 7 (1 ppm CPC/150 ppm mixed organic acids) was shown to completely inhibit the growth of Salmonella Enteritidis whereas 150 ppm of mixed acids alone (Formula 3) inhibited growth for only 12 h. Formula 6 (1 ppm CPC/100 ppm mixed acids) inhibit growth for 16 h with no inhibition noted for 100 ppm mixed acids alone. Essentially, 1 ppm CPC was able to substitute 50 ppm mixed acids. As described in Experiment 1, the combination of 1 ppm CPC/100 ppm propionic acid did not inhibit the growth of Salmonella Enteriditis, whereas in the current study the same combination with mixed organic acids controlled Salmonella for 12 h, confirming the well documented observation that mixed organic acids are generally more efficacious than a single organic acid. As expected 250 ppm mixed acid treatment alone completely inhibited the growth of Salmonella.

TABLE 9
CPC potentiation of mixed organic acids in inhibiting
Salmonella Enteriditis

| | Hours | | | | | | |
|---|---|---|---|---|---|---|---|
| | 0.000 | 4 | 8 | 12 | 16 | 20 | 24 |
| Treatment | Optical Density | | | | | | |
| PC | 0.000 | 0.025 | 0.264 | 0.392 | 0.413 | 0.372 | 0.333 |
| 1 | 0.000 | 0.014 | 0.216 | 0.38 | 0.442 | 0.447 | 0.410 |
| 2 | 0.000 | 0.003 | 0.102 | 0.296 | 0.405 | 0.455 | 0.452 |
| 3 | 0.000 | 0.001 | 0.001 | 0.008 | 0.048 | 0.153 | 0.296 |
| 4 | 0.000 | 0.001 | 0.000 | 0.000 | 0.000 | 0.000 | 0.000 |
| 5 | 0.000 | 0.003 | 0.138 | 0.320 | 0.388 | 0.422 | 0.415 |
| 6 | 0.000 | 0.000 | 0.000 | 0.000 | 0.009 | 0.128 | 0.295 |
| 7 | 0.000 | 0.000 | 0.001 | 0.000 | 0.000 | 0.003 | 0.009 |
| 8 | 0.000 | 0.000 | 0.000 | 0.000 | 0.000 | 0.000 | 0.000 |
| 9 | 0.000 | 0.000 | 0.000 | 0.000 | 0.000 | 0.000 | 0.000 |
| 10 | 0.000 | 0.000 | 0.000 | 0.000 | 0.000 | 0.000 | 0.000 |
| 11 | 0.000 | 0.000 | 0.000 | 0.000 | 0.000 | 0.000 | 0.000 |
| 12 | 0.000 | 0.000 | 0.000 | 0.000 | 0.000 | 0.000 | 0.000 |
| 13 | 0.000 | 0.000 | 0.000 | 0.000 | 0.000 | 0.000 | 0.000 |
| 14 | 0.000 | 0.000 | 0.000 | 0.000 | 0.000 | 0.000 | 0.000 |
| 15 | 0.000 | 0.000 | 0.000 | 0.000 | 0.000 | 0.000 | 0.000 |

In table 10 and FIG. 9, Formula 7 (1 ppm CPC/150 ppm mixed organic acids) was shown to completely inhibit the growth of E. coli O157:H7 whereas 150 ppm of mixed acids alone (Formula 3) inhibited growth for up to 6 h only. Formula 6 (1 ppm CPC/100 ppm mixed acids) was not effective inhibiting E. coli, but delayed its growth by about 4 hours compared with 100 ppm mixed acids alone (Formula 2). Although somewhat less effectively for E. coli than for Salmonella, again I ppm CPC was able to substitute 50 ppm of mixed organic acids. As anticipated 250 ppm acid treatment with no CPC (Formula 4) completely inhibited the growth of E. coli.

TABLE 10
CPC potentiation of mixed organic acids in inhibiting E. coli O157:h7

| | Hours | | | | | | |
|---|---|---|---|---|---|---|---|
| | 0 | 4 | 8 | 12 | 16 | 20 | 24 |
| Treatment | Optical Density | | | | | | |
| PC | 0.000 | 0.091 | 0.215 | 0.311 | 0.349 | 0.364 | 0.384 |
| 1 | 0.000 | 0.087 | 0.217 | 0.326 | 0.385 | 0.403 | 0.423 |
| 2 | 0.000 | 0.04 | 0.156 | 0.277 | 0.360 | 0.396 | 0.417 |
| 3 | 0.000 | 0.003 | 0.01 | 0.031 | 0.066 | 0.103 | 0.180 |
| 4 | 0.000 | 0.002 | 0.001 | 0.000 | 0.000 | 0.000 | 0.000 |
| 5 | 0.000 | 0.084 | 0.223 | 0.327 | 0.373 | 0.376 | 0.394 |
| 6 | 0.000 | 0.003 | 0.036 | 0.139 | 0.215 | 0.304 | 0.347 |
| 7 | 0.000 | 0.001 | 0.000 | 0.000 | 0.000 | 0.000 | 0.000 |
| 8 | 0.000 | 0.002 | 0.002 | 0.001 | 0.001 | 0.000 | 0.000 |
| 9 | 0.000 | 0.001 | 0.000 | 0.000 | 0.000 | 0.000 | 0.000 |
| 10 | 0.000 | 0.001 | 0.000 | 0.000 | 0.000 | 0.000 | 0.000 |
| 11 | 0.000 | 0.000 | 0.000 | 0.000 | 0.000 | 0.000 | 0.000 |
| 12 | 0.000 | 0.000 | 0.000 | 0.000 | 0.000 | 0.000 | 0.000 |
| 13 | 0.000 | 0.000 | 0.000 | 0.000 | 0.000 | 0.000 | 0.000 |
| 14 | 0.000 | 0.005 | 0.000 | 0.000 | 0.000 | 0.000 | 0.000 |
| 15 | 0.000 | 0.004 | 0.000 | 0.000 | 0.000 | 0.000 | 0.000 |

Low levels of CPC potentiate the antimicrobial power of mixed organic acids. These experiments resulted in the combination of 1 ppm CPC/150 ppm mixed organic acids effectively inhibiting the growth of two agricultural and food pathogens whereas 150 ppm mixed acids in the absence of CPC slowed growth but did not completely inhibit it. While this combination was not evaluated using propionic acid alone, it is expected based on previous data that these levels would be sufficient to inhibit the growth of S. Enteriditis. In the assay, a minimum of 250 ppm mixed organic acids without CPC was required for complete inhibition of both organisms.

As with the CPC/propionic acid combination, the modes of action of each molecule appear to be complementary. It is likely that the interaction of CPC with the bacterial cell membrane facilitates the entry of organic acids into the cell, resulting in a lower lethal dosage of the acids.

Experiment 4

Experiments were conducted to evaluate synergy between cetylpyridinium chloride (CPC) and acetic acid in the inhibition of microbial growth. A 3×4 matrix design combined low levels of CPC (0, 1, and 5 ppm) with acetic acid (50, 100, 150 and 250 ppm). Two microorganisms were evaluated via a microtiter plate assay. Low levels of CPC were found to potentiate the antimicrobial power of acetic acid. A greater effect was observed toward Staphylococcus aureus as quarternary ammonium compounds are very effective against Gram-positive bacteria.

Twelve formulations were prepared using the 3×4 matrix design in Table 11. Combinations were prepared using 0, 1, and 5 CPC and 50, 100, 150 and 250 ppm acetic acid and given numeric identifications of 1-12.

TABLE 11
A 3 × 4 matrix design of 12 combinations of
CPC and acetic acid and

Acetic Acid (ppm)

| CPC (ppm) | 50 | 100 | 150 | 250 |
|---|---|---|---|---|
| 0 | 1 | 2 | 3 | 4 |
| 1 | 5 | 6 | 7 | 8 |
| 5 | 9 | 10 | 11 | 12 |

Two test organisms—Salmonella Enteritidis (ATCC 13076) and Staphylococcus aureus (ATCC 25923) were selected for use in the initial screening assays. Cetylpyridinium chloride was obtained from Aceto Corporation. Acetic acid was obtained from Kemin Agri-Foods North America raw material inventory. S. Enteritidis and S. aureus were grown aerobically in Tryptic Soy Broth (TSB) for 24 h at 37° C. Test inocula were prepared to achieve a 10⁶ cfu/ml suspension of bacterial cells. A Petroff Hausser counting chamber was used to determine the level of inoculum.

To evaluate the efficacy of CPC and acetic acid combinations in bacterial inhibition, a microtiter plate assay was utilized. An Optimax microtiter plate reader (Molecular Devices, Sunnyvale, Calif.) at 405 nm wavelength was used to measure the optical density of the suspension in each well. Plates were read kinetically over 24 h. The temperature was maintained at 35° C. All results reflect the average optical density measurements of four microtiter wells. Treatments were prepared in sterile RO at the inclusion levels noted in Table 1. A 100 μl aliquot of test organism and a 100 μl aliquot of experimental treatment were dispensed into individual microtiter plate wells. Bacteria cultures were diluted in Nutrient Broth (NB) prior to inoculation into the wells due to cloudiness caused by the interaction of TSB medium and CPC. Positive controls consisting of 100 μl of test organism in NB and 100 μl of sterile water and a negative control consisting of 100 μl of NB (no organisms) and 100 μl of sterile water were also used.

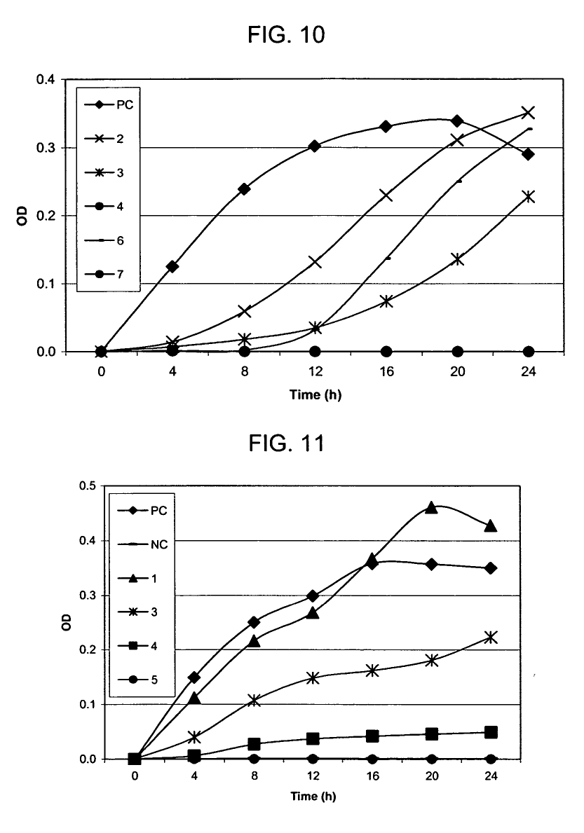

Cetylpyridinium chloride was found to potentiate the antimicrobial effect of acetic acid. Tables 12 and 13 represent the effects of CPC and acetic acid in the inhibition of two organisms. Selected data is illustrated in FIGS. 10 and 11, respectively. In Table 12, FIG. 10, Formula 7 (1 ppm CPC/150 ppm acetic acid) was shown to completely inhibit the growth of S. Enteritidis whereas 150 ppm of acetic acid alone (Formula 3) inhibited growth for only 4 h. Formula 6 (1 ppm CPC/100 ppm acetic acid) inhibited growth for 8 h with less than 4 h inhibition noted for 100 ppm acetic acid alone (Formula 2). At 250 ppm acetic acid treatment alone completely inhibited the growth of S. Enteritidis. Essentially, when combined with acetic acid, CPC was able to substitute acetic acid in a 1:100 ratio.

TABLE 12
CPC potentiation of acetic acid on the inhibition of S. Entertidis
measured by reduced optical density (OD)

| | Hours | | | | | | |
|---|---|---|---|---|---|---|---|
| | 0 | 4 | 8 | 12 | 16 | 20 | 24 |
| Formulation | Optical Density (OD) | | | | | | |
| PC¹ | 0.000 | 0.125 | 0.239 | 0.302 | 0.331 | 0.339 | 0.290 |
| NC² | 0.000 | 0.000 | 0.001 | 0.001 | 0.001 | 0.001 | 0.001 |
| 1 | 0.000 | 0.111 | 0.225 | 0.318 | 0.362 | 0.390 | 0.390 |
| 2 | 0.000 | 0.014 | 0.059 | 0.132 | 0.230 | 0.311 | 0.351 |
| 3 | 0.000 | 0.007 | 0.018 | 0.035 | 0.074 | 0.136 | 0.228 |
| 4 | 0.000 | 0.001 | 0.000 | 0.000 | 0.000 | 0.000 | 0.000 |
| 5 | 0.000 | 0.062 | 0.182 | 0.290 | 0.344 | 0.363 | 0.374 |
| 6 | 0.000 | 0.000 | 0.002 | 0.032 | 0.137 | 0.250 | 0.327 |
| 7 | 0.000 | 0.001 | 0.000 | 0.000 | 0.000 | 0.000 | 0.000 |
| 8 | 0.000 | 0.004 | 0.001 | 0.000 | 0.000 | 0.000 | 0.000 |
| 9 | 0.000 | 0.000 | 0.000 | 0.000 | 0.000 | 0.000 | 0.000 |
| 10 | 0.000 | 0.000 | 0.000 | 0.000 | 0.000 | 0.000 | 0.000 |
| 11 | 0.000 | 0.000 | 0.000 | 0.000 | 0.000 | 0.000 | 0.000 |
| 12 | 0.000 | 0.000 | 0.000 | 0.000 | 0.000 | 0.000 | 0.000 |

¹Positive Control
²Negative Control

In Table 13, FIG. 11, Formula 5 (1 ppm CPC/50 ppm acetic acid) was able to completely inhibit the growth of S. aureus whereas 50 ppm acetic acid (Formula 1) resulted in no inhibition at all. Even a level of 150 ppm acetic acid (Formula 3) did not inhibit growth and, in this experiment, minimal growth was observed at an exposure of 250 ppm acetic acid (Formula 4). These data support that CPC could substitute for acetic acid in an approximate ratio of 1:200.

These results support the mutual potentiation of CPC and organic acids as 2.5 ppm CPC alone did not inhibit either S. Enteritidis or S. aureus (see Experiment 1).

TABLE 13
CPC potentiation of acetic acid on the inhibition of S. aureus
measured by reduced optical density (OD)

| | Hours | | | | | | |
|---|---|---|---|---|---|---|---|
| | 0 | 4 | 8 | 12 | 16 | 20 | 24 |
| Formulation | Optical Density (OD) | | | | | | |
| PC¹ | 0.000 | 0.149 | 0.25 | 0.299 | 0.358 | 0.357 | 0.350 |
| NC² | 0.000 | 0.000 | 0.001 | 0.001 | 0.001 | 0.001 | 0.001 |
| 1 | 0.000 | 0.112 | 0.216 | 0.268 | 0.367 | 0.461 | 0.428 |
| 2 | 0.000 | 0.048 | 0.119 | 0.143 | 0.196 | 0.275 | 0.367 |
| 3 | 0.000 | 0.040 | 0.107 | 0.148 | 0.162 | 0.181 | 0.223 |
| 4 | 0.000 | 0.006 | 0.027 | 0.037 | 0.042 | 0.046 | 0.049 |
| 5 | 0.000 | 0.000 | 0.000 | 0.000 | 0.000 | 0.000 | 0.000 |
| 6 | 0.000 | 0.006 | 0.004 | 0.003 | 0.005 | 0.006 | 0.002 |
| 7 | 0.000 | 0.001 | 0.000 | 0.000 | 0.000 | 0.000 | 0.000 |
| 8 | 0.000 | 0.000 | 0.014 | 0.000 | 0.000 | 0.000 | 0.000 |
| 9 | 0.000 | 0.000 | 0.000 | 0.000 | 0.000 | 0.000 | 0.000 |
| 10 | 0.000 | 0.003 | 0.000 | 0.000 | 0.000 | 0.000 | 0.000 |
| 11 | 0.000 | 0.005 | 0.005 | 0.001 | 0.002 | 0.001 | 0.000 |
| 12 | 0.000 | 0.000 | 0.000 | 0.000 | 0.000 | 0.000 | 0.000 |

¹Positive Control
²Negative Control

Low levels of CPC potentiate the antimicrobial power of acetic acid. As observed with propionic acid, the modes of action of each molecule appear to be complementary. It is likely that the interaction of CPC with the bacterial cell membrane facilitates the entry of the organic acid into the cell, resulting in a lower lethal dosage. A higher degree of potentiation was attained in the inhibition of the Gram-positive organism. These experiments show that CPC in combination with acetic acid will augment the reduction of pathogens and enhance food safety.

Experiment 5

Animals are exposed to a variety of pathogenic organisms through their drinking water. These pathogens can be transferred via the animal to the processing plants elevating the incidences of food-borne illnesses in humans. Reducing the contamination levels of the drinking water can be accomplished by treatment with organic acids. The potentiation of mixed organic acids in the reduction of pathogens has been previously demonstrated by the inclusion of CPC. The presence of CPC allows for lower inclusion levels of organic acids. Experiments were conducted to evaluate cetylpyridinium chloride (CPC) and mixed organic acids in the reduction of coliforms in livestock drinking water. Farm drinking water was collected from three locations. Two test formulations were used at three inclusion levels.

Drinking water samples were received from three locations (a swine, a turkey, and a dairy farm, respectively) and held under refrigeration until tested. A phosphate stock solution was prepared by mixing 34 g KH₂PO₄ in 500 ml water, adjust to pH 7.2, and dilute to 1,000 ml. Butterfield's phosphate buffer diluent was prepared by adding 1.25 ml of the phosphate stock solution to 1,000 ml water, adding 1 drop of Tween-80, stirring well and sterilizing prior to use. An initial background coliform level was determined for each water source by serially diluting in the Butterfield's phosphate buffer diluent a 1-mL aliquot to its endpoint. The samples are plated with MacConkey II agar and incubated at 35° C. to 37° C. for 24 hours. Coliform colonies are those colonies that are brick red, dark pink, or dark purple due to fermentation of lactose in the media. Plates are selected from each dilution set that contain approximately 20 to 200 coliform colonies. Counts are averaged and, when multiplied by the dilution factor, yield the number of coliform colony forming units per gram of sample (cfu/g).

Treatment levels of 0, 520, 2600, and 5200 ppm (0, 0.52, 2.6, and 5.2 mL/L or 0.06, 0.33, and 0.66 oz/gal) of a commercial water acidifier, referred to herein as KS, were used. (The acidifier was KEM SAN™ Liquid available from Kemin Agrifoods North America, Des Moines, Iowa, which is a propionic acid-based, mixed organic acid acidifier with a label-recommended treatment level of 0.33-1 oz/gal). To evaluate the ability to decrease the water acidifier usage by inclusion of CPC, the following treatment levels of CPC/acidifier were used: 326, 1630, and 3260 ppm—which assumes a 1:100 CPC:mixed acid substitution as determined by previous experimentation. For the purposes of the current experiments, a CPC inclusion rate of 1:100 was used (designated 1×) as well as 1:50 (designated 2×) and 1:33 (designated 3×).

One hundred (100) mL samples of water were tested. All samples were held at room temperature throughout the experiment. At Oh, 2 h and 6 h post treatment, samples were thoroughly mixed, duplicate 1-mL aliquots were sampled, and serially diluted in phosphate buffer to their endpoint, then plated with MacConkey II agar and incubated at 37° C. for 24 h, followed by coliform enumeration.

The first experiment used three water samples treated at three levels with either KS water acidifier alone or KS with 3×CPC inclusion. Untreated water samples ranged from 10² cfu/ml to 10⁶ cfu/ml of coliforms, providing a valid product challenge at various contamination levels. Table 14 reflects the ability of each treatment to affect coliform levels at various concentrations. Site 1 offered a considerable microbial challenge, enabling the determination of CPC's ability to potentiate the antimicrobial activity of mixed organic acids. Both treatments reduced coliform levels as compared to the untreated water. However, at both 2 and 6 h, the CPC/KS treatments were more effective (P=0.025) in reducing the level of coliforms in water at 1630 and 3260 ppm levels than KS at 2600 and 5200 ppm levels. Neither site 2 nor site 3 provided much challenge and both treatments were capable of completely eliminating coliforms from the water at either the mid or high treatment levels. Minimal effect on coliform levels was observed at the lowest treatment level of either product, although application of 326 ppm of the CPC/KS mix did eliminate all coliforms in the water from site 3 as opposed to 520 ppm/KS.

TABLE 14
Coliform levels (cfu/ml) over time in water treated with KS water acidifier alone
or KS with 3X CPC. Results reflect the average of duplicate assays.

| | Site 1 | | | Site 2 | | | Site 3 | | |
|---|---|---|---|---|---|---|---|---|---|
| | Time (h) | | | | | | | | |
| Treatment | 0 | 2 | 6 | 0 | 2 | 6 | 0 | 2 | 6 |
| Control | 2800000 | 1500000 | 1900000 | 1100 | 1300 | 2700 | 660 | 570 | 1300 |
| KS | | | | | | | | | |
| 520 ppm | NA | 1200000 | 600000 | NA | 2100 | 900 | NA | 500 | 500 |
| 2600 ppm | NA | 450000 | 150000 | NA | 0 | 0 | NA | 0 | 0 |
| 5200 ppm | NA | 200000 | 180000 | NA | 0 | 0 | NA | 0 | 0 |
| KS w/CPC | | | | | | | | | |
| 326 ppm | NA | 2600000 | 900000 | NA | 1800 | 3900 | NA | 0 | 0 |
| 1630 ppm | NA | 65000 | 20000 | NA | 0 | 0 | NA | 0 | 0 |
| 3260 ppm | NA | 15 | 0 | NA | 0 | 0 | NA | 0 | 0 |

NA = not applicable

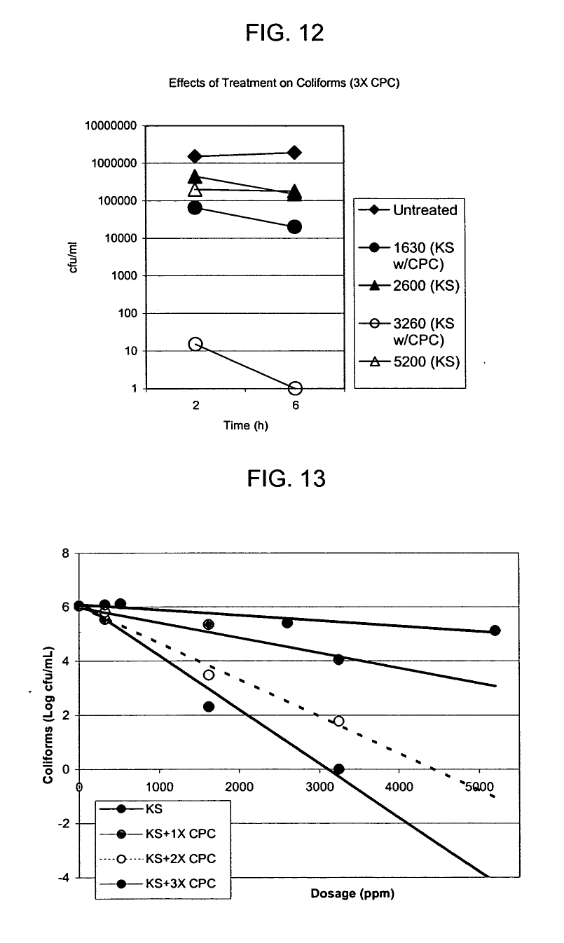

As illustrated in FIG. 12, the formulation containing 30 ppm CPC and 1600 ppm KS (total 1630 ppm) reduced the level of coliforms in Site I water by 2 logs compared to the untreated water, whereas 2600 ppm KS reduced the number of coliforms by 1 log. At twice the application rate (3260 ppm), the combination treatment caused a dramatic reduction in coliform level, by 5 logs at 2 h and 6 logs at 6 h, compared to untreated water. The comparable KS treatment of 5200 ppm reduced coliform levels by only 1 log after either 2 h or 6 h. In other words, the CPC containing formula resulted in a highly effective dose response in sanitizing water from site 1, as opposed to the formula without CPC. In addition, a more pronounced reduction in counts was noted between 2 h and 6 h for the highest application rate of the formula containing CPC.

Table 15 contains the results of a second, more detailed experiment challenging water from Site 1 with KS water acidifier containing 1, 2 or 3×levels of CPC, compared with KS water acidifier without CPC. As observed in the previous experiment, the lowest treatment level of either product was not effective in reducing the level of coliforms. However, KS with either 2× or 3×CPC outperformed KS alone at both the mid and high treatment levels with KS w/2×CPC or 3×CPC more efficacious at the 1630 ppm level than KS at 5200 ppm.

TABLE 15
Coliform levels (cfu/ml) over time in water from site 1 treated with
KS alone or KS with 1, 2 or 3X CPC (KS w/CPC). Results reflect
the average of duplicate assays.

| | Component | | | | |
|---|---|---|---|---|---|
| | (ppm) | | Time (h) | | |
| Treatment | KS | CPC | 0 | 2 | 6 |
| Control | | | 450000 | 400000 | 1100000 |
| KS | | | | | |
| 520 ppm | 520 | 0 | NA | 250000 | 1300000 |
| 2600 ppm | 2600 | 0 | NA | 150000 | 250000 |
| 5200 ppm | 5200 | 0 | NA | 100000 | 130000 |
| KS | | | | | |
| w/1XCPC | | | | | |
| 326 ppm | 324 | 2 | NA | 200000 | 350000 |
| 1630 ppm | 1620 | 10 | NA | 550000 | 220000 |
| 3260 ppm | 3240 | 20 | NA | 500000 | 11000 |
| KS | | | | | |
| w/2XCPC | | | | | |
| 326 ppm | 322 | 4 | NA | 250000 | 650000 |
| 1630 ppm | 1610 | 20 | NA | 50000 | 3000 |
| 3260 ppm | 3220 | 40 | NA | 3500 | 60 |
| KS | | | | | |
| w/3XCPC | | | | | |
| 326 ppm | 320 | 6 | NA | 250000 | 1200000 |
| 1630 ppm | 1600 | 30 | NA | 7500 | 200 |
| 3260 ppm | 3200 | 60 | NA | 0 | 0 |

The formulations including CPC outperformed KS alone in reducing coliforms in highly contaminated drinking water. CPC at 2× reduced coliforms by nearly 1 log at 2 h over untreated water and a total reduction of nearly 3 logs by 6 h. CPC at 3× reduced coliforms by nearly 2 logs at the 2 h time period as compared to untreated water with a total reduction of approximately 4 logs at 6 h. KS alone at 2600 ppm showed no reduction in coliform counts at 2 h compared to untreated water with close to a 1 log reduction at 6 h. Again, the CPC containing formulas displayed a clear dose response, which was slightly more pronounced (P<0.05) for the 1× formula vs KS alone, but much more pronounced (P<0.01) for the 2× and 3× formulas compared with the 1× formula (FIG. 13).

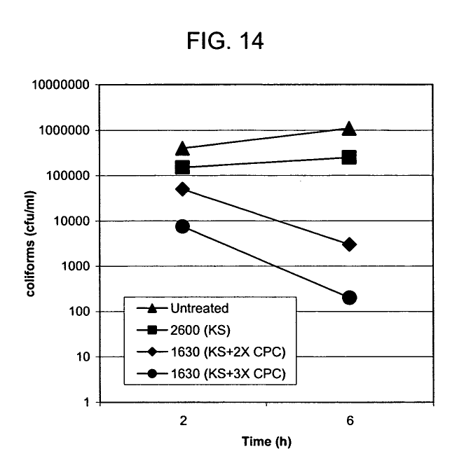

Further, a larger reduction in counts was noted between 2 h and 6 h for the formulas containing CPC than for KS alone (FIG. 14).

The ability of mixed organic acids to reduce the level of pathogenic bacteria in practical livestock drinking water can be drastically improved with the inclusion of CPC. These experiments have shown that mixed organic acids in combination with CPC are more effective in reducing coliform levels and this improvement occurs at much lower treatment levels than mixed organic acids alone.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

1. A method of reducing the level of pathogens in water or inhibiting the growth of pathogens and spoilage organisms in water, comprising the step of adding to the water an effective amount of a quaternary ammonium compound selected from the group consisting of alkylpyridinium, tetra-alkylammonium, and alkylalicyclic ammonium salts.

2. The method of claim 1, wherein the amount of the quaternary ammonium compound applied is between about 0.1 part per million and about 1000 parts per million.

3. The method of claim 1, wherein the quaternary ammonium compound is cetylpyridinium chloride.

4. The method of claim 3, wherein the amount of cetylpyridinium chloride applied is between about 1.0 part per million and about 100 parts per million.

5. A method of reducing the level of pathogens in water or inhibiting the growth of pathogens and spoilage organisms in water, comprising the steps of adding to the water an organic acid or blend of organic acids selected from the group consisting of acetic, benzoic, citric, formic, fumaric, lactic, propionic, and sorbic acids and one or more quaternary ammonium compounds selected from the group consisting of alkylpyridinium, tetra-alkylammonium, and alkylalicyclic ammonium salts.

6. The method of claim 5, wherein the amount of the quaternary ammonium compound applied is between about 0.1 part per million and about 1000 part per million and the organic acid compound applied is between about 5 part per million and about 10,000 part per million.

7. The method of claim 5, wherein the quaternary ammonium compound is cetylpyridinium chloride.

8. The method of claim 7, wherein the amount of cetylpyridinium chloride applied is between about 1.0 part per million and about 100 part per million and the organic acid compound applied is between about 25 part per million and about 2500 part per million.

9. A method of reducing the amount of organic acids required to reduce the level of pathogenic microorganisms or inhibit the growth of spoilage microorganisms in water, comprising the step of substituting between about 10 percent and about 95 percent of the organic acid with between about 0.1 weight percent and about 10 weight percent of one or more quaternary ammonium compounds selected from the group consisting of alkylpyridinium, tetra-alkylammonium, and alkylalicyclic ammonium salts.

10. An antimicrobial wash, comprising water which has been treated according to the method of claim 5 wherein the quaternary ammonium compound is present at a level between about 0.1 part per million and about 1000 part per million and the organic acid compound applied is between about 5 part per million and about 10,000 part per million.

11. The antimicrobial wash of claim 10, wherein the quaternary ammonium compound is cetylpyridinium chloride.

12. The antimicrobial wash of claim 11, wherein the amount of cetylpyridinium chloride applied is between about 1.0 part per million and about 100 part per million and the organic acid compound applied is between about 25 part per million and about 2500 part per million.

13. A method of reducing the pathogenic or spoilage organism load of a product, comprising the step of applying the antimicrobial wash of claim 10, and wherein the products are selected from the group consisting of fruits, vegetables, meat, and animal carcasses.
